(19) United States
(12) Patent Application Publication
(10) Pub. No.: US 2024/0123011 A1
(43) Pub. Date: Apr. 18, 2024

(54) COMPOSITION CONTAINING BLACK GINSENG AS ACTIVE INGREDIENT FOR PREVENTION, ALLEVIATION, OR TREATMENT OF CORONAVIRUS INFECTION

(71) Applicant: GINSENG BYPHARM CO., LTD., Gangwon-do (KR)

(72) Inventor: Junmin AN, Gangwon-do (KR)

(21) Appl. No.: 18/275,643

(22) PCT Filed: Feb. 3, 2022
(86) PCT No.: PCT/KR2022/001709

§ 371 Date: Aug. 3, 2023

(30) Foreign Application Priority Data
Feb. 9, 2021 (KR) 10-2021-0018096

(51) Int. Cl. A61K 36/258; A61P 31/14

(52) U.S. Cl. CPC A61K 36/258; A61P 31/14; A61K 2236/19; A61K 2236/33; A61K 2236/331

(57) ABSTRACT

A pharmaceutical composition for prevention, palliation, or treatment of coronavirus infection and a food composition for prevention or alleviation of coronavirus infection, each composition containing black ginseng or a black ginseng extract as an active ingredient is provided. The composition exhibits excellent antiviral activity against corona viruses, which cause respiratory diseases, gastrointestinal diseases, liver diseases, brain diseases, etc., especially against corona virus-19 and thus, can be advantageously used in medications and foods for prevention, alleviation, or treatment of diseases caused by corona virus infection.

### BACKGROUND

The present invention relates to a black ginseng composition for prophylaxis, alleviation or treatment of coronavirus infection, more specifically, a composition for prophylaxis, alleviation or treatment of coronavirus infection using black ginseng, a black ginseng extract, an enzyme-treated product of the extract, and or a fermented product of the extract as an active ingredient.

Coronaviruses are RNA viruses that cause respiratory, digestive, liver, and brain diseases in mammals and birds (Gallagher T M. et. al., Virology, 279(2):371-374, 2001). In particular, among viruses belonging to the coronavirus, transmissible gastroenteritis virus (TGEV) and porcine epidemic diarrhea virus (PEDV) are highly contagious viruses that invade the gastrointestinal tract, cause dehydration and fever due to vomiting and diarrhea, and have a high mortality rate, resulting in considerable economic loss (Duarte M. et. al., J Gen Virol., 75 (Pt 5):1195-1200, 1994).

Although these viruses have a very high mortality rate, there is no reliable treatment for diseases caused by other viral infections.

Coronavirus disease 2019 or COVID-19 caused by coronavirus first emerged in Wuhan, China in December 2019 and has since spread throughout China and around the world, which is a respiratory infection caused by a new type of coronavirus (SARSCoV-2; RNA virus belonging to Coronaviridae).

Initially, it was known only as a respiratory infectious disease of unknown cause, however on Jan. 9, 2020, the pathogen was identified as the cause of the pneumonia was a new type of coronavirus (SARS-CoV-2, named by the International Committee on Classification of Viruses on February 11).

The pathogen of COVID-19 is “SARS-CoV-2”. The International Committee on Taxonomy of Viruses (ICTV) published a paper on Feb. 11, 2020, proposing the name SARS-CoV-2 for the pathogen of COVID-19. According to ICTV, this name emphasized that the virus is similar to SARS (Severe Acute Respiratory Syndrome), which was prevalent in 2003.

Korea obtained and analyzed the gene sequence of the virus disclosed by China through academia, and as a result, it was confirmed that it had the highest homology (89.1%) with a similar bat-derived coronavirus. The homology with the four human coronaviruses was as low as 39% to 43%, while 50% for MERS and 77.5% for SARS.

Coronavirus disease 2019 (COVID-19), which is currently a worldwide issue, is defined as a respiratory syndrome caused by infection with a new coronavirus (severe acute respiratory syndrome coronavirus 2, SARS-CoV-2). The disease classification of COVID-19 is the Class 1 Infectious Disease, “Emerging Infectious Disease Syndrome”, with the disease code of U07.1.

The pathogen is SARS-CoV-2, an RNA virus belonging to Coronaviridae, and the transmission route is droplet (saliva droplet) or contact, as identified so far. In most cases, infection is transmitted through droplets produced when coughing or sneezing, or by touching objects contaminated with the coronavirus and then touching the eyes, nose, or mouth.

The global fatality rate due to coronavirus infection averages about 3.4% (based on WHO, 3.5). Currently, 6% of infected people worldwide are dead, so the level of fatality rate by country and age is very different. This virus causes severe illness and death primarily in the elderly, immunocompromised patients, and those with underlying medical conditions.

Coronaviruses (CoVs) are RNA viruses with a helical structure of a single positive strand and consists of a total of 5 structural proteins. Due to the double spike glycoprotein (S protein), they look like a crown or halo on an electron microscope (Schoeman and Fielding, 2019). CoVs have the largest known genomes among RNA viruses (27-34 kb), and are subdivided into 4 groups of alpha, beta, delta, and gamma, among which the beta variant is highly contagious and virulence.

SARS-CoV-2 invades by binding its surface S protein to angiotensin converting enzyme 2 (ACE2) on host cells, and ACE2 is highly distributed in the oral and nasal mucosa, nasopharynx, lungs, lungs, stomach, small intestine, and large intestine, characterized by the findings of viral pneumonia (Hamming et al., 2004).

The incubation period for coronavirus is 1 to 14 days (average 4 to 7 days). The presence of coronavirus is diagnosed by isolating the virus from the sample or detecting a specific gene in the sample according to the test criteria for diagnosis.

Symptoms of COVID-19 include various respiratory infections ranging from mild to severe, such as fever, malaise, cough, shortness of breath, and pneumonia, as well as sputum, sore throat, headache, hemoptysis, nausea, and diarrhea. Currently, there is no specific antiviral agent other than conservative treatments such as fluid replacement and antipyretics. The World Health Organization (WHO) announced that there is no evidence that having antibodies to SARS-CoV-2 can protect against reinfection and infection with COVID-19 disease, which highlights the importance of drug development.

### SUMMARY OF THE INVENTION

The inventors, while studying the effectiveness of black ginseng, found that when black ginseng had advantageous effects of inhibiting the replication and infection of coronavirus, as it had showed inhibition against infection and replication of SARS-CoV-2 virus in Vero-E6 cells, body temperature increase and weight loss in hamsters infected with SARS-CoV-2, and significantly SARS-CoV-2 spike protein binding activity in hamsters infected with SARS-CoV-2, thereby achieving the present invention. Therefore, an object of the present invention is to provide a composition for prophylaxis, alleviation or treatment of coronavirus infection, containing black ginseng as an active ingredient.

To achieve the object above, the present invention provides a pharmaceutical composition or health food composition for prophylaxis, alleviation or treatment of coronavirus infection, preferably COVID-19 virus (SARS-CoV-2) infection, containing at least one active ingredient selected from the group consisting of black ginseng, a black ginseng extracts and mixtures thereof.

In the composition according to the invention as described above, the black ginseng extract may be extracted using water or ethanol as the extraction solvent.

In the composition according to the invention as described above, the black ginseng extract may be extracted and then treated with one or more enzymes selected from the group consisting of β-galactosidase, β-glucosidase, β-glucanase, α-amylase, and cellulase.

In the composition according to the invention as described above, the black ginseng extract is fermented with lactic acid bacteria or yeast.

In the composition according to the invention as described above, the black ginseng extract is treated with enzymes and then fermented with lactic acid bacteria or yeast.

The composition of the present invention exhibits excellent antiviral activity against coronaviruses, particularly COVID-19 viruses, which are viruses that cause respiratory diseases, digestive diseases, liver diseases and brain diseases, and thus may be useful in medicaments and food products for prophylaxis, alleviation or treatment of diseases caused by coronavirus infection.

### BRIEF DESCRIPTION OF DRAWINGS

FIG. 1 shows the inhibition of coronavirus infection in Vero-E6 host cells using a composition of the present invention.

FIG. 2 shows viral replication inhibitory effect on Vero-E6 host cells infected with coronavirus using the composition of the present invention.

FIG. 3 shows the effect of inhibiting SARS-CoV-2 spike protein binding activity in hamsters infected with SARS-CoV-2 using the composition of the present invention.

### DETAILED DESCRIPTION OF THE INVENTION

The present invention relates to a composition for prophylaxis, alleviation or treatment of coronavirus infection using black ginseng, a black ginseng extract, an enzyme-treated product of black ginseng extract, or a fermented product of black ginseng extract, as an active ingredient. Significant outcomes were confirmed in experiments on inhibition against infection and replication of SARS-CoV-2 virus in Vero-E6 cells using the composition of the present invention. The composition of the present invention drastically decreased the spike protein binding activity of SARS-CoV-2 in a coronavirus-infected hamster model. Inhibition of body temperature increase and weight loss of experimental animals were also confirmed. Accordingly, the composition of the present invention has been shown to inhibit the cellular infection of coronavirus through inhibition of the spike protein binding activity of SARS-CoV-2, suggesting that it is effective as a composition for prophylaxis, alleviation or treatment of coronavirus infection with excellent therapeutic effect but no toxicity.

Therefore, the present invention provides a pharmaceutical composition for prophylaxis or treatment of coronavirus infection containing a black ginseng extract as an active ingredient.

Herein the term “coronavirus” may be any one or more selected from the group consisting of transmissible gastroenteritis virus (TGEV), porcine epidemic diarrhea virus (PEDV), canine coronavirus, and bovine coronavirus, SARS coronavirus (SARS-CoV) and COVID-19 virus (SARS-CoV-2).

Further, the present invention provides a health functional food composition for prophylaxis or alleviation of coronavirus infection containing a black ginseng extract as an active ingredient.

Black ginseng used in the present invention may be used without particular limitation as long as it is widely known in the art: black ginseng powder, an extract obtained by solvent extraction of black ginseng, preferably an extract obtained by using water or ethanol as an extraction solvent, and a mixture thereof.

An extraction method of the present invention can be used without particular limitation as long as it is widely known in the art: for example, heat extraction, cold extraction, reflux cooling extraction, steam distillation, ultrasonic extraction, elution and compression. Depending on the purpose, the extract may be subjected to a conventional fractionation process and may be purified according to a conventional purification.

Further, powder may be prepared by pulverizing the extracted primary extract through an additional process such as distillation under reduced pressure and freeze drying or spray drying. A purified fraction may be obtained through various chromatography methods such as silica gel column chromatography, high performance liquid chromatography, and thin layer chromatography.

Accordingly, it is understood that the extract includes all extracts, separated compounds, fractions and purified products obtained in each step of extraction, fractionation or purification, as well as dilution, concentration and dried products thereof in the present invention.

In the present invention, the black ginseng extract may be used after being treated with an enzyme or fermented, or may be used after being treated with an enzyme and then fermented.

The enzyme used in the present invention is preferably one or more selected from the group consisting of β-galactosidase, β-glucosidase, β-glucanase, α-amylase, and cellulase.

The strain used for fermentation of the present invention may use any of all strains known to ferment black ginseng, and may preferably be lactic acid bacteria or yeast.

Specifically, the preparation of the black ginseng extract used in the present invention includes:
an extraction step of extracting black ginseng with hot water at 70 to 100° C., or alternatively, an extraction step of extracting black ginseng with 70% by weight ethanol;
an enzyme treatment step of adding an enzyme to the extract of the extraction step and performing enzyme treatment at 25 to 65° C. for 2 to 7 hours; and
a fermentation step of adding lactic acid bacteria or yeast, preferably Saccharomyces cerevisiae, to the enzyme-treated extract and fermenting at 20 to 40° C. for 10 to 48 hours.

Hereinafter, the configuration and effects of the present invention will be described in more detail referring to examples and test examples. These examples and test examples are only for illustrating the present invention, but the scope of the present invention is not limited thereto.

### Example: Preparation of Sample

As the black ginseng extract, a hot water extract, an ethanol extract, an enzyme-treated product treated with α-amylase after extraction, and an enzyme-treated product with α-amylase after extraction and then treated with Saccharomyces cerevisiae were used.

In particular, the extracts were prepared by:
an extraction step of extracting black ginseng with hot water at 70 to 100° C., or alternatively, an extraction step of extracting black ginseng with 70% by weight ethanol;
an enzyme treatment step of adding an enzyme to the extract of the extraction step and performing enzyme treatment at 45° C. for 3 hours; and
a fermentation step of adding Saccharomyces cerevisiae to the enzyme-treated extract and fermenting at 30° C. for 24 hours.
Sample 1: Black ginseng hot water extract
Sample 2: Black ginseng ethanol extract
Sample 3: Black ginseng hot water extract subject to enzyme treatment
Sample 4: black ginseng ethanol extract subject to enzyme treatment
Sample 5: Fermented black ginseng hot water extract
Sample 6: Fermented black ginseng ethanol extract

### Test Example 1: Virus Infection Inhibitory Effect

Vero-E6 cells were cultured in DMEM (−/−) and inoculated at a concentration of 5×10⁵ cells/well in 12 well-plates for virus infection experiments. After culturing for 24 hours at 37° C. in a CO₂ incubator, the cell medium was removed and washed twice with 1×PBS.

After pre-treatment with the sample at a concentration of 100 μg/ml and incubation at 37° C. in a CO₂ incubator for 2 hours, the cell medium was removed for viral infection, and then the SARS-CoV-2 virus was treated at a concentration of 50 pfu/well for 1 hour. The cell medium was removed, 1.5 ml of the medium was added to each well, and the reaction was performed for 72 hours to confirm the results. At this time, 5 μM of Remdesivir (Rem), which is known to have a therapeutic effect on the SARS-CoV-2 virus, and 10 μM of chloroquinone phosphate (CP), a malaria treatment, were set as control groups.

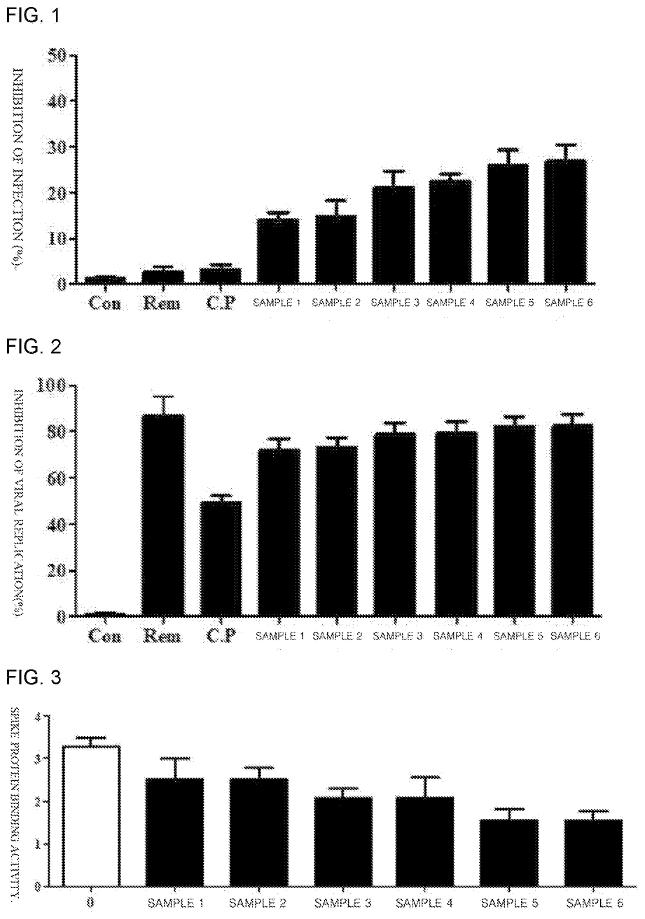

As shown in FIG. 1, all samples according to the present invention exhibited excellent viral infection inhibitory effects, and in particular, showed much better results than those of the control groups, remdesivir or chloroquinone phosphate.

Referring to FIG. 1, the samples treated with enzymes and the fermented samples showed more excellent effects, and there was no significant difference between the hot water extract and the ethanol extract.

### <Test Example 2. Inhibitory Effect of Viral Replication of Black Ginseng Extract

Vero-E6 cells were cultured in DMEM (—/—) and inoculated at a concentration of 5×10⁵ cells/well in 12 well-plates for virus infection experiments. After culturing for 24 hours at 37° C. in a CO₂ incubator, the cell medium was removed and washed twice with 1×PBS. For viral infection, the SARS-CoV-2 virus was treated for 1 hour at a concentration of 50 pfu/well, then 1.5 ml of medium containing 100 μg/ml of the sample was added to each well, reacted for 72 hours, and the results were confirmed.

At this time, 5 μM of Remdesivir (Rem), which is known to have a therapeutic effect on the SARS-CoV-2 virus, and 10 μM of chloroquinone phosphate (CP), a malaria treatment, were set as control groups. The results are shown in FIG. 2.

As shown in FIG. 2, all samples according to the present invention exhibited excellent virus replication inhibitory effect against SARS-CoV-2, and exhibited antiviral effect without cytotoxicity. It showed improved results compared to chloroquinone phosphate, and almost the same results as remdesivir.

Referring to FIG. 2, the samples treated with enzymes and the fermented samples showed more excellent effects, and there was no significant difference between the hot water extract and the ethanol extract.

### Test Example 3: Antiviral Effect

Six-week-old hamsters were obtained, randomly grouped, and then acclimated for one week. Hamsters were infected with SARS-CoV-2, and after 24 hours, each sample was orally administered at a concentration of 100 mg/kg for 4 days. For 4 days before and after virus administration, body weight was measured every day, and body temperature was measured once before and after infection.

On Day 4 after virus administration, the saliva was collected from all hamsters and subjected to pretreatment, and the antiviral effect was measured by confirming the binding activity of the spike protein using the SARS-CoV-2 Spike RBD Nanobody kit. The results are shown in FIG. 3.

As shown in FIG. 3, the samples according to the present invention showed the effect of inhibiting the binding activity of viruses. In particular, the samples treated with enzymes and the fermented samples showed more excellent effects, and there was no significant difference between the hot water extract and the ethanol extract.

### Test Example 4: Antipyretic Effect and Weight Loss Inhibitory Effect

COVID-19 is accompanied by fever due to viral infection. Accordingly, in Test Example 3, each hamster's body temperature was measured on Day 0 and Day 4 of infection to measure the effect of alleviating the symptoms of COVID-19. The results are shown in Table 1.

TABLE 1

Body Temperature (° C.)
Period of Infection (Days)

| | Day 0 | |
|---|---|---|
| | (Pre-infection) | Day 4 |
| Control Groups | 39.10 ± 1.756 | 42.52 ± 1.119 |
| Sample 1 | 39.08 ± 1.714 | 39.54 ± 1.712 |
| Sample 2 | 39.10 ± 1.725 | 39.54 ± 1.744 |
| Sample 3 | 39.07 ± 1.734 | 39.11 ± 1.388 |
| Sample 4 | 39.09 ± 1.756 | 39.11 ± 1.341 |
| Sample 5 | 39.10 ± 1.755 | 39.08 ± 1.289 |
| Sample 6 | 39.10 ± 1.752 | 39.08 ± 1.356 |

As shown in Table 1, although the body temperature increased due to coronavirus infection, it was confirmed that the increase in body temperature was suppressed due to the administration of the samples according to the present invention. In particular, the samples treated with enzymes and the fermented samples showed more excellent effects, and there was no significant difference between the hot water extract and the ethanol extract.

COVID-19 causes weight loss due to viral infection. Accordingly, in Test Example 3, the weight loss inhibitory effect was measured by measuring the weight of hamsters a total of 5 times from Day 0 to Day 4 of infection. The results are shown in Table 2.

TABLE 2

| | Body Temperature (° C.) | | | | |
|---|---|---|---|---|---|
| | Period of Infection (Days) | | | | |
| | Day 0 | | | | |
| | (Pre-infection) | Day 1 | Day 2 | Day 3 | Day 1 |
| Control | 109.5 ± 1.85 | 107.5 ± 1.84 | 106.3 ± 1.71 | 105.7 ± 1.14 | 105.2 ± 1.59 |
| Groups | | | | | |
| Sample 1 | 109.8 ± 1.75 | 109.2 ± 1.55 | 109.5 ± 1.73 | 109.5 ± 1.26 | 109.6 ± 1.13 |
| Sample 2 | 109.9 ± 1.88 | 109.2 ± 1.75 | 109.6 ± 1.82 | 109.6 ± 1.24 | 109.7 ± 1.35 |
| Sample 3 | 109.9 ± 1.49 | 109.5 ± 1.23 | 109.8 ± 1.71 | 109.9 ± 1.15 | 109.9 ± 1.25 |
| Sample 4 | 109.9 ± 1.38 | 109.5 ± 1.25 | 109.9 ± 1.15 | 109.9 ± 1.16 | 109.9 ± 1.26 |
| Sample 5 | 109.9 ± 1.72 | 109.6 ± 1.38 | 109.9 ± 1.36 | 109.9 ± 1.85 | 109.9 ± 1.48 |
| Sample 6 | 109.9 ± 1.74 | 109.6 ± 1.83 | 109.9 ± 1.31 | 109.9 ± 1.85 | 109.9 ± 1.45 |

As shown in Table 2, although the body weight loss occurred due to coronavirus infection, it was confirmed that the body weight loss was suppressed due to the administration of the samples according to the present invention. In particular, the samples treated with enzymes and the fermented samples showed more excellent effects, and there was no significant difference between the hot water extract and the ethanol extract.

Through the above test examples, the black ginseng extract of the present invention showed significant infection and replication inhibitory effects against the SARS-CoV-2. Further, when infecting the hamster with the coronavirus, it was confirmed that there is an excellent antiviral efficacy based on the body weight, body temperature, and SARS-CoV-2 spike protein binding ability.

Referring to the above test examples, the black ginseng extract is predicted to inhibit the coronavirus by inhibiting RNA replication and protein binding ability of SARS-CoV-2.

Therefore, the composition according to the present invention exhibits excellent antiviral activity against coronavirus, especially the COVID-19 virus causing respiratory diseases, digestive diseases, liver diseases, and brain diseases. Thus the composition can be usefully used in medicines and foods for prophylaxis, alleviation or treatment of diseases caused by coronavirus infection.

1. A pharmaceutical composition for prophylaxis, alleviation or treatment of coronavirus infection, the composition comprising at least one selected from the group consisting of black ginseng, a black ginseng extract, and mixtures thereof as an active ingredient.

2. The composition according to claim 1, wherein the coronavirus is COVID-19 virus (SARS-CoV-2).

3. The composition according to claim 1, wherein the coronavirus is COVID-19 virus (SARS-CoV-2).

4. The composition according to claim 1, wherein the black ginseng extract is extracted using water or ethanol as an extraction solvent.

5. The composition according to claim 4, wherein the black ginseng extract is extracted and then treated with any one or more enzymes selected from the group consisting of β-galactosidase, β-glucosidase, β-glucanase, α-amylase and cellulase.

6. The composition according to claim 4, wherein the black ginseng extract is fermented with lactic acid bacteria or yeast after being extracted.

7. The composition according to claim 5, wherein the black ginseng extract is fermented with lactic acid bacteria or yeast after being treated with enzymes.

8. The composition according to claim 2, wherein the coronavirus is COVID-19 virus (SARS-CoV-2).

9. The composition according to claim 2, wherein the black ginseng extract is extracted using water or ethanol as an extraction solvent.

10. The composition according to claim 9, wherein the black ginseng extract is extracted and then treated with any one or more enzymes selected from the group consisting of β-galactosidase, β-glucosidase, β-glucanase, α-amylase and cellulase.

11. The composition according to claim 9, wherein the black ginseng extract is fermented with lactic acid bacteria or yeast after being extracted.

12. The composition according to claim 10, wherein the black ginseng extract is fermented with lactic acid bacteria or yeast after being treated with enzymes.
